(19) United States
(12) Patent Application Publication
(10) Pub. No.: US 2024/0016599 A1
(43) Pub. Date: Jan. 18, 2024

(54) IMPLANTABLE DEVICE, IN PARTICULAR AN IMPLANTABLE BREAST DEVICE

(71) Applicant: LATTICE MEDICAL, LOOS (FR)

(72) Inventors: Jaime Andres DESTOUESSE VILLA, LILLE (FR); Shengheng CAO, LILLE (FR)

(21) Appl. No.: 18/252,175

(22) PCT Filed: Nov. 8, 2021
(86) PCT No.: PCT/EP2021/080987

§ 371 Date: May 8, 2023

(30) Foreign Application Priority Data
Nov. 9, 2020 (FR) 2011492

(51) Int. Cl. A61F 2/12; A61L 27/58; A61L 27/56; A61L 27/18

(52) U.S. Cl. CPC A61F 2/12; A61L 27/58; A61L 27/56; A61L 27/18

(57) ABSTRACT

The present invention relates to an implantable device, in particular an implantable breast device, comprising a main shell and a chamber for treating at least one soft tissue, the device being further configured to receive at least one vascular pedicle, the main shell comprising a set of through-openings and being bioresorbable. Advantageously, the main shell comprises at least one bioresorbable (co)polymer having an elongation at break greater than or equal to 200%.

### CROSS-REFERENCE TO RELATED APPLICATIONS

This application is a national phase entry of International Patent Application No. PCT/EP2021/080987 (filed 8 Nov. 2021), which claims priority to French Patent Application No. 2011492 (filed 9 Nov. 2020), the entire disclosures of which are incorporated herein by reference.

### BACKGROUND

### Technical Field

The present invention relates to an implantable device, in particular an implantable breast device, and to a method for manufacturing said device.

An object of the present invention is an implantable device for replacing and/or increasing a volume of soft tissue, in particular following the removal of a volume of tissue from the diseased subject.

### Prior Art

A breast implant can be implanted in order to increase the volume of a breast without the breast presenting any particular pathology, or for reconstruction of the latter following removal of a given volume of diseased tissue.

There are currently many types of breast implant. Breast implants formed from a pouch filled with a silicone gel have the disadvantage that the pouch can rupture, releasing the silicone gel. Furthermore, the silicone-filled pouch is permanently implanted in the body.

Implantable devices described in WO 2018/078489 are known, comprising a shell delimiting a determined volume in which a soft tissue will grow, said shell receiving at least one vascular pedicle and a volume of soft tissue, such as a volume of adipose tissue, disposed on the vascular pedicle. The vascular pedicle enables the vascularisation, and hence the supply to the adipose tissue which grows and fills the shell. The shell comprises a plurality of through-openings which allow the volume of soft tissue to be supplied with serous fluid rich in growth factors. It is also envisaged that the lateral through-openings for placing a pedicle are sufficient on their own for supplying the soft tissue with serous fluid. The shell can also be a profiled element with spider legs (FIG. 10), thus enabling a good supply of serous fluid, but not satisfactorily constraining the soft tissue growth volume. Moreover, this profiled element does not have satisfactory mechanical properties.

This technique has the advantage that the volume of the chest is reconstructed in a single surgical operation. The soft tissue develops gradually, at the same time as the resorption of the resorbable shell. Moreover, the implanted autologous soft tissue comprises a volume of fat, and not muscle, such as the latissimus dorsi muscle, for example. The removal of the soft tissue is less invasive for the patient, and the surgical consequences are therefore less burdensome.

There is, nevertheless, a need to improve the growth of the soft tissue housed in the shell, in particular its homogeneous supply with serous fluid that is rich in growth factors.

There is also a need for an implantable device, in particular an implantable breast device, that is reliably and totally reabsorbed at the end of an agreed period.

There is also a need to reduce the quantity of implanted prosthetic materials, while providing an implantable device that is sufficiently mechanically resistant, and ensuring its primary function of homogeneous soft tissue growth.

Finally, there is a need for an implantable device improving patient comfort.

### BRIEF DESCRIPTION

The present invention relates, according to a first aspect, to an implantable device comprising a main shell and a chamber for treating at least one soft tissue, the device being further configured to receive at least one vascular pedicle, the main shell comprising a set of through-openings and being bioresorbable. Advantageously, said main shell comprises at least one bioresorbable (co)polymer having an elongation at break greater than or equal to 200%.

The combination of a very flexible bioresorbable polymer or copolymer, having an elastic behaviour, with a main shell partially or totally delimiting a treatment chamber and comprising a set of through-openings, enables a main shell to be formed which is highly resistant to compression, in a durable manner.

Advantageously, the main shell withstands a compression fatigue test comprising 325,000 cycles, each cycle corresponding to a compression force of 50 newtons, applied at a frequency of 3.3 Hz. This number of cycles is estimated as corresponding to maintaining mechanical properties over a duration of 6 months. This number of cycles is extrapolated from a breast implant compression fatigue test method comprising the application of 6 million cycles estimated as corresponding to maintaining mechanical properties over a period of 10 years.

The treatment chamber is preferably a tissue engineering chamber.

The main shell is preferably substantially dome-shaped.

In an embodiment, a first manual pressure applied on the apex of the main shell, in particular towards the base of the main shell, causes a deformation thereof, said main shell then recovers its initial shape, free of any stress or under the effect of a second manual pressure applied opposite to said first manual pressure.

Advantageously, the main shell is sufficiently flexible to be able to be deformed which facilitates its insertion at the site to be treated, and it possible to reduce the size of the incision made on the patient for its insertion, for example to approximately 4 cm to 5 cm.

In an embodiment, the bioresorbable (co)polymer has an elongation at break (%) greater than or equal to 300%, preferably greater than or equal to 400%, more preferably greater than or equal to 500%, preferably greater than or equal to 700%, in particular greater than or equal to 900%, in particular measured in the printing directions, X or Y, in three dimensions of the tested specimen.

In an embodiment, the bioresorbable (co)polymer has an elongation at break (%) less than or equal to 2000%, preferably less than or equal to 1500%, more preferably less than or equal to 1200%, in particular measured in the printing directions, X or Y, in three dimensions of the tested specimen.

In an embodiment, the bioresorbable (co)polymer has a Young's modulus greater than or equal to 100 MPa, in particular greater than or equal to 150 MPa (preferably whatever the printing directions in three dimensions of the tested specimen: X, Y, Z).

In an embodiment, the bioresorbable (co)polymer has a Young's modulus less than or equal to 600 MPa, in particular less than or equal to 400 MPa (preferably whatever the printing directions in three dimensions of the tested specimen: X, Y, Z).

The Young's modulus and the elongation at break are measured according to standard ASTM D638-14 (“Standard Test Method for Tensile Properties of Plastics”) at a traction speed of 10 mm/min on V-type specimens, preferably at a temperature of 20° C. The specimens tested are preferably manufactured by an additive manufacturing process, in particular the printing direction can vary along the axes X, Y or Z.

The main shell preferably comprises an outer surface and an inner surface oriented facing the treatment chamber, in particular the inner surface is substantially opposite the outer surface.

Preferably, the through-openings emerge on the outer and inner surfaces of the main shell and extend between said outer and inner surfaces.

Preferably, the through-openings emerge in the volume of the treatment chamber and on the outside of the main shell, in particular they are arranged in its main wall, in particular having a dome shape.

In an embodiment, the through-openings, designated in the present document, have a single same shape or have a plurality of different combined shapes, in particular said one or more shapes can be chosen from: a round shape, a parallelepiped shape such as a square or a rectangle, a triangle shape, a random cell shape, preferably they have a substantially round shape.

In an embodiment, the through-openings, in particular substantially circular through-openings, have at least one dimension, in particular a diameter, greater than or equal to 1 mm, in particular less than or equal to 15 mm, more particularly less than or equal to 10 mm, in particular between 3 mm and 6 mm.

In an embodiment, the through-openings comprise through-openings for which the distance separating substantially circular and adjacent through-openings, aligned along an axis A1, is between 4 mm and 8 mm or between 5 mm and 7 mm, and the distance separating substantially circular and adjacent through-openings, aligned along an axis A2 secant with the axis A1 and forming an angle of approximately 45°, is between 6 mm and 10 mm or between 7 mm and 9 mm.

Preferably, the main shell has a main wall having a thickness ecp greater than 0 mm, and less than or equal to 8 mm, more preferably less than or equal to 5 mm, in particular less than or equal to 3 mm.

Preferably, the thickness of the main wall of the main shell is substantially constant.

Preferably, the main shell has a substantially hemispherical shape, such as a dome, and comprises a base having a predetermined diameter and a predetermined height (likewise corresponding to the depth of the main shell).

In an embodiment, the base of the main shell has a diameter greater than or equal to 7 cm and less than or equal to 16 cm, in particular greater than or equal to 9 cm and less than or equal to 14 cm, in particular between 100 mm and 130 mm (including upper and lower limits).

In an embodiment, the height of the main shell is greater than or equal to 1 cm, in particular greater than or equal to 3 cm, and less than or equal to 6 cm.

The main shell exists in different sizes, and is chosen depending on the dimensions of the final targeted reconstruction.

In an embodiment, the main shell has a mass greater than or equal to 10 g, preferably less than or equal to 100 g, more preferably less than or equal to 50 g.

The mass (g) is preferably determined in the present text at a temperature greater than or equal to 20° C. and less than or equal to 25° C.; in particular at atmospheric pressure, for example of 1 atm, and in particular with a relative humidity of 50%.

In an embodiment, the main shell has a volume greater than or equal to 150 cm³, preferably less than or equal to 500 cm³.

Volume of the main shell (or of the intermediate shell described below), shall mean its internal volume, delimited by the inner surface of the main or intermediate shell.

Advantageously, within the context of the present invention, it is understood that the internal volume of the main shell, or the volume of the treatment chamber, is empty. This provision makes it possible to place one or more vascular pedicles and/or one or more materials (for example one or more textile layers or 3D printed layers) in said internal volume of the main shell or in said volume of the treatment chamber.

By definition, the main shell and/or the intermediate shell is/are hollow, in particular the main shell and/or the intermediate shell comprise an internal volume or treatment chamber that can receive one or more vascular pedicles and/or one or more fat layers and/or one or more porous layers (in particular synthetic), in particular textile or 3D-printed.

In an embodiment, the volume of the treatment chamber is substantially equal to the internal volume delimited by the main shell and optionally the main bottom, and optionally supplemented by the internal volume delimited by the intermediate shell.

In an embodiment, the main shell has a reaction force to static compression greater than or equal to 400 N (in particular for a main shell for which the volume is greater than or equal to 175 cm³ and less than or equal to 475 cm³), in particular greater than or equal to 800 N for a main shell for which the volume is greater than or equal to 475 cm³.

In an embodiment, the main shell does not exhibit a break after a shock of 4.4 kg, repeated at least 3 times.

In an embodiment, the main shell does not exhibit a break after 325,000 compression fatigue cycles in a humid environment at 37° C., each cycle comprising the application of a force of 50 Newtons at 3.3 Hz. This value is in particular valid for a main shell for which the volume is between 150 cm³ and 500 cm³.

The static compression reaction force (N), the impact behaviour and the resistance to compression fatigue are preferably measured according to standard ISO 14607:2018 (entitled: non-active surgical implants—mammary implants—Particular requirements).

A control main shell was made from a polymer of lactic acid and glycolic acid (PLGA (by weight): 85:15) with an elongation at break much less than 200%, in particular of order 5 to 10%. Said control main shell, the volume of which is of order 200 cm³ or 300 cm³, cracks or even breaks during the impact test and the above-mentioned compression fatigue test between 20,000 and 50,000 cycles.

The main shell according to the invention, despite its shape, its low weight and the large number of through-openings, has good mechanical performance obtained by using a highly flexible and bioresorbable (co)polymer.

In an embodiment, said at least one bioresorbable (co)polymer can be a copolymer or a terpolymer.

Said (co)polymer preferably comprises at least two different repeat units.

Said (co)polymer is preferably a polymer of ε-caprolactone and at least one repeat unit different from ε-caprolactone, for example from lactic acid and/or glycolic acid.

Preferably, the repeat units from lactic acid can be L-lactic acid repeat units and/or D-lactic acid repeat units and/or DL-lactic acid repeat units.

Preferably, said at least one (co)polymer comprises ε-caprolactone repeat units, the molar fraction of which (in said (co)polymer) is less than or equal to 50%, more preferably less than or equal to 40%, in particular between 20% and 40% (limits included).

Preferably, said at least one (co)polymer comprises L-lactic acid repeat units and/or D-lactic acid repeat units and/or DL-lactic acid repeat units, the molar fraction of which (in said (co)polymer) is greater than or equal to 50%, more preferably greater than or equal to 60%, in particular between 60% and 80% (limits included).

Said molar fractions can be determined by NMR spectroscopy.

Preferably, said at least one (co)polymer comprises L-lactic acid repeat units and/or, D-lactic acid repeat units and/or DL-lactic acid repeat units, the molar fraction of which is less than or equal to 90%, more preferably less than or equal to 80%.

In an embodiment, the intermediate shell and/or the main bottom described below each comprises said at least one bioresorbable (co)polymer.

Preferably, the mass fraction of said at least one bioresorbable (co)polymer in the main shell (in other words, relative to the total mass of the main shell), and/or in the main bottom (in other words relative to the total mass of the main bottom), and/or in the intermediate shell (in other words relative to the total mass of the intermediate shell), is greater than or equal to 50%, more preferably greater than or equal to 80%, preferably greater than or equal to 90%, in particular of order 100%.

In an embodiment, the main shell, and optionally the main bottom and/or the intermediate shell, substantially consist of said at least one resorbable (co)polymer.

Preferably, the main shell delimits an inner space forming at least one portion, or substantially all, of the treatment chamber. The main shell thus protects the growth of the soft tissue. The volume of the inner space, at least partially defined by the main shell, is gradually filled as the soft tissue grows. The growth and therefore the expansion of the tissue are thus controlled by the volume of the treatment chamber. The treatment chamber thus allows the growth of cells capable of forming a soft tissue, this new soft tissue is formed in situ in the living organism in which the device is implanted in order to replace and/or increase a volume of tissue of the body of the subject.

The soft tissue comprises at least one layer of cells chosen from adipocytes, cells capable of differentiating into adipocytes, and mixtures of these two types of cells.

In the present document, “soft tissue” shall be understood to mean adipose tissue, and more particularly deep adipose tissue, taken for example by liposuction.

The above-mentioned cells are preferably obtained by using the supernatant after centrifugation of a fatty mass taken from the subject herself. It is an autologous graft, limiting rejection reactions.

In the present document, “cells capable of differentiating into adipose cells” shall mean adult stem cells, in particular adult mesenchymal stem cells (i.e. coming from an adult subject) which are able to differentiate into cells that can differentiate into adipocytes.

It also possible that the implantable device comprises at least one porous layer combined with at least one layer of cells chosen from adipocytes. Said porous layer can be an assembly of at least two stacked layers, said layers being obtained by entanglement of yarns and/or fibres connected at connection points. Said at least one layer of cells chosen from adipocytes is preferably an intermediate layer disposed between said at least two porous layers. Said at least one porous layer can be chosen from textiles (knitted, fabric, non-woven), for example can be electrospun, or be printed in three dimensions, or a combination thereof.

Said at least one porous layer combined with at least one layer of cells chosen from adipocytes is preferably disposed in the treatment chamber, in particular in the upper treatment chamber and/or in the lower treatment chamber (cf. below).

In the present document, “connection points” is understood to mean all the points of contact between yarns and/or fibres, these contact points being obtained mechanically (i.e. by knitting, weaving, needling, etc.) or chemically (by fusing together, for example, one or more yarns and/or fibres, and/or by bonding using a binder, for example).

Preferably, the main shell, and more preferably, the main bottom and/or the upper receiving partition and/or the intermediate shell and/or the lateral partition, and/or the one or more yarns and/or the fibres, are each made of one or more bioresorbable materials.

The one or more bioresorbable materials, including said at least one bioresorbable (co)polymer, are, as well as the configuration of the main shell and/or the main bottom and/or the intermediate shell, and the through-cells, preferably determined so as to obtain a bio-resorption, and in particular of the implantable device, at the end of 6 months or, for example, at the end of 12 months or 18 months.

Preferably, the one or more bioresorbable materials, in particular said at least one bioresorbable (co)polymer, are chosen from the list comprising: polyesters, in particular aliphatic polyesters, for example polydioxanone, or, in particular, polymers from the copolymerisation of at least one monomer chosen from: a glycolic acid monomer, an L-, D-, or DL-lactide monomer; an ε-caprolactone monomer; or a mixture thereof; more preferably from a polymer of L-lactic acid (PLLA) or D-lactic acid (PLDA) or DL-lactic acid (PDLLA) or a mixture thereof; a glycolic acid polymer (PGA); a copolymer of L-, D-DL-lactic acid and glycolic acid; a copolymer of ε-caprolactone and L-, D-, DL-lactide.

Preferably, said at least one (co)polymer has a glass transition temperature below 50° C., more preferably between 25° C. and 40° C., in particular measured at the rate of 10° C./min according to standard ISO 11357-1: 2016 and USO 11357-2:2020 (entitled Plastics—Differential scanning calorimetry (DSC)—Part 2: Determination of glass transition temperature and step height).

In the present document, “resorbable” or “bioresorbable” are understood to mean any object (material, shell, etc.) having the property of degrading when it is implanted in the body of a living subject, the degradation products being removed by the organism of the living subject in such a way that said object has been totally removed at the end of a predetermined period (depending on the nature, quantity and structure of the object, in particular), for example after of order 3 months or 6 months or 18 months.

Preferably, the main shell, and/or the intermediate shell, comprises at least one lateral through-opening, optionally two lateral through-openings, forming at least one through-passage enabling the insertion of at least one vascular pedicle. A portion of the vascular pedicle is thus placed in the treatment chamber by passing through a lateral through-opening, and partially in the inner space of the main or intermediate shell, and another portion of said vascular pedicle projects to the outside of the main or intermediate shell. The vascular pedicle thus serves as intermediate supply element between the region of the body receiving the implantable device, and the cells of the soft tissue disposed in the treatment chamber.

Preferably, the one or more lateral through-openings are one or more access windows to the internal volume of the main shell, or of the intermediate shell. The one more lateral through-openings have at least one dimension that is larger, in particular at least 3 times or 5 times larger, than one of the dimensions of said through-openings.

In the present document, “inner surface” shall mean any surface oriented towards the treatment chamber. Conversely, in the present document, “outer surface” shall mean any surface oriented to the outside of the main shell or of the intermediate shell or of the main bottom described below.

In an alternative, said bioresorbable (co)polymer is a copolymer of ε-caprolactone and of L-, D-of DL-lactide.

The Applicant has determined that this polymer can attain the targeted mechanical performance in the context of the present invention, in particular fatigue resistance and impact resistance.

Said copolymer can be in the form of poly(L-lactide or D-lactide or DL-lactide-co-ε-caprolactone), in particular poly(L-lactide-co-ε-caprolactone).

In an alternative, said bioresorbable (co)polymer has a number average molar mass Mn greater than or equal to 10,000 g/mol, preferably greater than or equal to g/mol, more preferably greater than or equal to 45,000 g/mol, in particular greater than or equal to 65,000 g/mol.

In an alternative, said bioresorbable (co)polymer has a number average molar mass Mn less than or equal to 200,000 g/mol, preferably less than or equal to 175,000 g/mol, more preferably less than or equal to 150,000 g/mol, in particular less than or equal to 120,000 g/mol or 100 00 g/mol.

In an alternative, said bioresorbable (co)polymer has a mass average molar mass Mw greater than or equal to 50,000 g/mol, preferably greater than or equal to g/mol, more preferably greater than or equal to 100,000 g/mol, in particular greater than or equal to 125,000 g/mol.

In an alternative, said bioresorbable (co)polymer has a number average molar mass Mw less than or equal to 300,000 g/mol, preferably less than or equal to 250,000 g/mol, more preferably less than or equal to 200,000 g/mol, in particular less than or equal to 180,000 g/mol or 100 00 g/mol.

In an alternative, the polydispersity index of said at least one bioresorbable (co)polymer is less than or equal to 2.5 or 2.3 or 2 or 1.8.

In an alternative, the polydispersity index (I=Mw/Mn) of the bioresorbable (co)polymer is greater than or equal to 1 or 1.3 or 1.5.

In an alternative, the main shell has an outer surface, and the ratio of the total surface area (mm²) of the through-openings, over the total surface area of the outer surface of the main shell, is greater than or equal to 35%, preferably less than or equal to 60%.

Advantageously, the mass of the main shell is optimised in order that it satisfies the targeted mechanical performance while enabling the resorption time to be controlled, and improving patient comfort.

Moreover, this porosity connected to the through-cells promotes the homogeneous circulation of serous fluid rich in growth factors towards the soft tissue to be developed.

The structure of the main shell, and optionally the structure of the intermediate shell, advantageously provide sufficient mechanical properties to maintain a volume once implanted that is substantially equal to the volume of the treatment chamber, at least during the period of bio-resorption. This provision advantageously makes it possible to also obtain an immediate aesthetic reconstruction for the subject, in a single surgical operation.

The main shell, and optionally the intermediate shell, must however have a porosity connected to the through-openings greater than a certain threshold in order that it has sufficient mechanical properties, and this during the possible resorption of the main shell, in order to orient, and protect, the development of the soft tissue.

Preferably, the total surface area and the open surface area of the main shell or of the intermediate shell are estimated by means of the design software SOLIDWORKS 2019-SPO4 (the procedure used is: 1/tab “evaluate”, 2/Select the surface, 3/option “measurement”, 4/surface value).

Preferably, the total surface area (mm²) of the through-cells emerging on the outer surface of the main shell is substantially equal to the total surface area of the through-cells emerging on the inner surface of the main shell. The thickness of the main wall delimiting said through-openings is therefore substantially constant.

Preferably, the surface area of the outer and/or inner surfaces of the main shell, the intermediate shell, the through-cells the side wall, and the upper receiving wall (below), is measured by considering these parts when flat, having a thickness substantially equal to 2 mm.

In an alternative embodiment, the main shell has an outer surface, and the ratio of the surface area of the solid outer surface of the main shell, over the total surface area of the outer surface of the main shell, is greater than or equal to 40%, preferably greater than or equal to 50%, in particular less than or equal to 70%.

The term “solid outer surface” is understood to mean the surface occupied by the structure or the skeleton of the main or intermediate shell.

In an alternative embodiment, the skeleton of the main shell has a fill ratio greater than or equal to 70%, more preferably greater than or equal to 80%, more preferably greater than or equal to 90%, in particular of order 100%.

When the fill ratio is for example of order 70%, this means that the void content in the skeleton of the main shell is of order 30%.

This fill ratio also preferably applies to the skeleton of the intermediate shell and/or to the skeleton of the main bottom.

This provision advantageously makes it possible to improve the mechanical properties of the main shell and/or the intermediate shell and/or the main bottom.

Preferably, the main shell and/or the intermediate shell and/or the main bottom is manufactured by an additive manufacturing process enabling the fill ratio to be regulated. The assembly of through-openings provides the necessary porosity for the implant. It is not necessary to further increase the porosity by having a low filling rate, which improves the mechanical performance.

In an alternative embodiment, the device comprises a main bottom having through-openings and forming a rear portion of the treatment chamber.

The main shell preferably forms a front portion of the treatment chamber.

In an embodiment, the main bottom comprises an inner face (in particular facing the treatment chamber) and an outer face, opposite the inner face, said through-openings extend between said inner and outer faces and emerge thereon.

At least one flap of soft tissue is thus maintained in the treatment chamber, between the main bottom and the main shell. Access of fat and/or unwanted tissues into the treatment chamber is thus prevented.

Preferably, the main bottom has a mass greater than 0 g and less than or equal to 20 g, more preferably less than or equal to 12 g.

The main bottom is preferably bioresorbable.

In an alternative embodiment, the device comprises an intermediate shell comprising through-openings and an upper receiving partition subdividing the treatment chamber into an upper treatment chamber and a lower treatment chamber, said device being configured so that each of said upper and lower treatment chambers is able to receive at least one vascular pedicle.

The intermediate shell is preferably bioresorbable.

This provision advantageously enables the volume of the treatment chamber to be increased by spacing apart the one or more soft tissues disposed in the upper treatment chamber from that or those disposed in the lower treatment chamber. It is thus possible to promote a reconstruction of a volume of order 500 cm³ to 700-800 cm³. Indeed, in order to reconstruct a volume of soft tissue having a significant volume, for example of 400 cm³ and more, the inventors have discovered that it is preferable to subdivide this volume, in order to form upper and lower treatment chambers, each preferably receiving an assembly of porous layers with an assembly of layers of cells chosen from adipocytes, said assembly being connected to and supplied by at least one vascular pedicle.

In an embodiment, the intermediate shell has a frustoconical shape.

In an embodiment, the upper receiving partition comprises an inner face (in particular oriented facing the treatment chamber) and an outer face, substantially opposite the inner face, and said through-openings of the intermediate shell extend between the inner and outer faces of the upper receiving partition and emerge thereon.

In an alternative embodiment, said upper receiving partition is spaced apart from the main bottom by a distance d greater than 0 mm in order to at least partially define the lower treatment chamber.

In an alternative embodiment, the intermediate shell comprises a lateral partition projecting from a lower face of the upper receiving wall, in particular into the lower treatment chamber, more particularly projecting towards the main bottom.

Preferably, the lateral partition comprises an inner surface and an outer surface, and through-openings extending between said inner and outer surfaces, and emerging thereon.

Preferably, the ratio of the total surface area (mm²) of the through-openings emerging on the outer surface of the lateral partition, over the surface area of the outer surface of the lateral partition is greater than or equal to 40%, preferably less than or equal to 60%, more preferably less than or equal to 50%.

The main wall of the lateral partition preferably has a thickness ecl greater than 0 mm, and less than or equal to 5 mm, more preferably less than or equal to 3 mm.

The total surface area (mm²) (and thus the size) of through-openings emerging on the outer surface of the lateral partition is substantially equal to the total surface area of the through-openings emerging on the inner surface of the peripheral rim. The thickness of the main wall delimiting said through-openings is therefore substantially constant.

The skeleton of the intermediate shell preferably has a fill ratio greater than or equal to 70%, more preferably greater than or equal to 80%, more preferably less than or equal to 90%.

In an alternative, the main bottom comprises one or more recesses, open towards the treatment chamber, and configured to receive, in particular by interlocking, a portion or portions of a lower edge, in particular of an annular lower edge, of the main shell or intermediate shell.

This provision facilitates the securing of the main bottom two the main or intermediate shell.

In an alternative, the main bottom comprises:
a continuous or discontinuous outer rim, projecting from an inner face of the main bottom into the treatment chamber, in particular into the lower treatment chamber, in particular along the peripheral edge of the main bottom, and—one or more portions of vertical wall projecting from the inner face of the main bottom into the treatment chamber, and at least partially delimiting with said outer rim, said one or more recesses.

In an alternative, the main shell or the intermediate shell comprises an outer lower edge and an inner lower edge set back from said outer lower edge.

Preferably, the inner lower edge is configured to cooperate, by interlocking, with said one or more receiving recesses of the main bottom.

In an alternative, the outer lower edge abuts on the outer rim of the main bottom.

In an alternative, the main shell and/or the intermediate shell each comprises one or more securing openings, in particular adjacent to the outer lower edge, and the main bottom comprises one or more securing openings, in particular adjacent to the peripheral edge of said main bottom, said securing openings being configured so as to enable the passage of at least one securing member, such as a suture yarn, through a securing opening of the main shell and a securing opening of the main bottom.

Said securing openings are preferably through-openings, as defined in the present document.

The various ratios indicated in the present document with regard to the through-openings all comprise openings passing through the main or intermediate shell or the main bottom extending between the outer and inner surfaces.

In an alternative embodiment, at least some of the through-openings of the main shell and/or of the intermediate shell and/or of the main bottom are Voronoi polyhedra.

The through-openings are advantageously distributed along the outer surface (and therefore on the inner surface) of the main shell, and optionally the outer surface and the inner surface of the intermediate shell, according to the Voronoi diagram.

The inventors have discovered that this provision makes it possible to optimise the total surface area of the through-openings, as well as the mechanical properties of the main shell, and/or the intermediate shell.

Indeed, the distribution and size of said through-openings are determined randomly, the stresses exerted will therefore likewise be randomly distributed over the implant during its stressing.

The length, width and number of Voronoi cells are preferably defined in the Python programming language comprising a library dedicated to Voronoi cells, then the profile obtained for said cells is projected onto the main shell or the intermediate shell in order to define the through-openings according to the invention.

In an alternative embodiment, the main shell comprises at least two vertical arches, in particular extending from an apex of the main shell, and transverse sections, in particular at least partially annular, extending between said vertical arches and connected with the latter.

The free distal ends of the vertical arches are preferably connected by a base transverse section that is annular, at least in portions.

Preferably, the base transverse section is in a support plane.

Preferably, the transverse sections are arranged according to assemblies of transverse sections, the transverse sections of an assembly being disposed substantially in a transverse plane, said assemblies being disposed in transverse planes that are substantially parallel to one another, and in particular that substantially parallel to the support plane.

This provision can also be found in the intermediate shell. In this case, the vertical arches extend from the periphery of the upper receiving wall.

In an alternative embodiment, the main bottom and/or the upper receiving partition, which is in particular substantially flat, each comprises through-openings arranged in order to define a plurality of first patterns connected to one another by first connection structures.

In an alternative embodiment, the first patterns comprise one or more segments, in particular at least three segments and at most six segments, and at least one or more segments of each first pattern is secured to a first connection structure.

Preferably, said segments are substantially parallel to one another, and spaced apart from one another.

Preferably, the one or more segments (each) have a width less than or equal to 3 mm, in particular less than or equal to 2 mm, more particularly greater than or equal to 0.10 mm, in particular greater than or equal to 0.30 mm.

Preferably, the one or more segments (each) have a length less than or equal to 20 mm, in particular less than or equal to 15 mm, more particularly greater than or equal to 3 mm, in particular greater than or equal to 6 mm.

Preferably, the distance separating two adjacent segments of a first pattern is greater than or equal to 0.1 mm and less than or equal to 2 mm.

In an alternative embodiment, the first connection structures are each connected to at least three distinct first patterns.

In an alternative embodiment, the main bottom and/or the upper receiving partition each comprise, second connection structures, different from the first connection structures, each of the second connection structures being connected to at least two segments of a same first pattern.

In an alternative embodiment, at least one second connection structure is connected to at least three distinct first patterns. Preferably, all of the second connection structures are connected to at least three distinct first patterns.

In an alternative embodiment, the main shell and/or the intermediate shell each comprises one or more through and transverse openings, emerging into the treatment chamber, and enabling the placing of at least one vascular pedicle.

These transverse openings are preferably considered as through-openings, such as defined in the present document, for the calculation of the porosity.

In an alternative embodiment, the main shell is substantially dome-shaped.

In an alternative, the at least one of said main shell, said intermediate shell, said main bottom, and said upper receiving partition, or a combination of these, is obtained by additive or subtractive manufacturing, preferably additive manufacturing.

An additive manufacturing step is understood to mean any manufacture by addition or agglomeration of one or more materials, in particular bioresorbable materials, and this by addition of successive layers of material or materials under the control of an application member controlled by a computer program implemented by a computer (for example selective laser sintering, in other words a powder melted or sintered using the energy of a high-power laser, such as a CO₂ laser).

Preferably, the one or more skeletons of the main shell and/or of the intermediate shell and/or of the main bottom, and/or of the upper receiving wall and/or of the side wall each have a fill ratio greater than or equal to 70%, more preferably greater than or equal to 80%, more preferably greater than or equal to 90%, in particular of order 100%.

An object of the present invention, according to a second aspect, is a method for manufacturing an implantable device, in particular according to any of the alternative embodiments with reference to the first aspect of the invention, and said method comprises an additive or subtractive manufacturing step, preferably additive, of said main shell, and optionally of said intermediate shell and/or of the main bottom, with at least one yarn comprising said (co)polymer having an elongation at break greater than or equal to 200%.

Preferably, the mass fraction of said at least one bioresorbable (co)polymer as defined in said at least one yarn is greater than or equal to 80% or 90%, in particular of order 100%.

Another object of the present invention, according to a third aspect, is a method for ex-vivo manufacturing of an implantable device for replacing and/or treating a volume of soft tissue comprising:
providing an implantable device according to any one of the alternative embodiments with reference to the first aspect of the invention, and
the provision, in the treatment chamber, of cells chosen from adipocytes, cells capable of differentiating into adipocytes and the mixtures of these two types of cells, said cells preferably coming from said subject.

The alternative embodiments, as well as the definitions and embodiments according to a first aspect of the invention can be independently combined with one another and with the alternatives according to a second aspect and/or a third aspect of the invention.

### BRIEF DESCRIPTION OF THE DRAWINGS

The invention will be better understood upon reading the following description of exemplary embodiments of the invention, given solely as non-limiting examples and with reference to the attached drawings, wherein:

FIG. 1 shows schematically, in perspective view, the main shell of a first example of an implantable device according to the invention;

FIG. 2 shows schematically, in perspective view, the main bottom of the first example of an implantable device of FIG. 1;

FIG. 3 shows schematically, viewed from above, the main bottom of the first example of an implantable device of FIGS. 1 and 2;

FIG. 4 shows schematically, in perspective, a second example of an implantable device according to the invention;

FIG. 5 shows schematically, in perspective, the main shell of the second example of an implantable device shown in FIG. 4;

FIG. 6 shows schematically, viewed from above, the main bottom of the second example of an implantable device shown in FIGS. 4 and 5;

FIG. 7 shows schematically an enlargement of the geometric patterns of the main bottom shown in FIG. 6;

FIG. 8 shows schematically an alternative of the main shell of the first or second example of an implantable device according to the invention; and

FIG. 9 shows schematically an alternative of the main bottom of the first or second example of an implantable device according to the invention.

### DETAILED DESCRIPTION

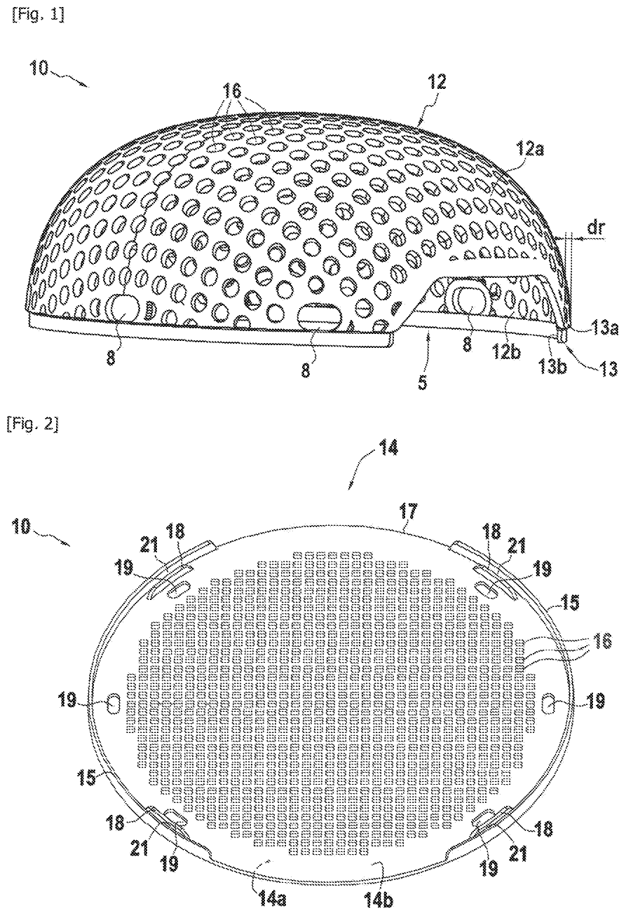

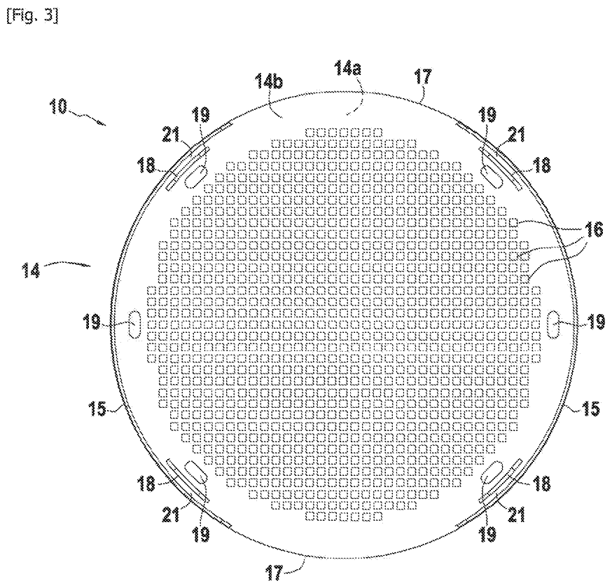

The first example of an implantable device 10 according to the invention comprises a main shell 12 shown in FIG. 1 and a main bottom 14, each provided with through-openings 16. The main shell 12, when it cooperates with the main bottom 14, defines a treatment chamber 5 configured to receive at least one soft tissue. The main bottom 14 forms a rear portion of the implantable device 10 and the main shell 12 forms a front portion of the implantable device 10. The main shell 12 and the main bottom 14 are bioresorbable, and each comprise at least one bioresorbable (co)polymer having an elongation at break greater than or equal to 200%, in this specific example of order 900-1000% in the directions X and Y of the tested specimen, in particular of type V, according to standard ASTM D638.14 at a traction speed of 10 mm/min. The Young's modulus of said polymer is preferably greater than or equal to 150 MPa, whether in the X, Y or Z directions of the specimen, in particular of type V, according to standard ASTM D638.14 at a traction speed of 10 mm/min.

In this specific example, the main shell 12 and the main bottom 14 are each obtained by an additive manufacturing method, by the use of a monofilament made of said flexible polymer, in particular of a copolymer of poly(L-lactide-co-ε-caprolactone), for which the molar fraction of ε-caprolactone is between 25% and 35%.

The main shell 12 and the main bottom 14 comprise through-openings 16 in order to promote exchanges of serous fluid and other growth factors, between the environment of the device 10 once implanted and the treatment chamber 5.

The main shell 12 has an outer surface 12a and an inner surface 12b, the ratio of, the total surface area (mm²) of the through-openings 16, over the total surface area (mm²) of the outer surface 12a of the main shell 12, is greater than or equal to 35% and less than or equal to 60%, in particular between 40% and 45%. This ratio is equivalent to a degree of opening.

The ratio of the surface area of the solid outer surface 12a of the main shell 12, over the total surface area of the outer surface 12a of the main shell 12, is greater than or equal to 40% and less than or equal to 70%, in particular between 55% and 60%. This ratio is equivalent to the degree of closure.

The same degree of opening as that given above applies to the main bottom 14 for its outer face 14a or its inner face 14b, in other words of order 40% to 45%. The same degree of closure as that given above applies to the main bottom 14 for its outer face 14a or inner face 14b, in other words of order 55% to 60%.

The main shell 12 and the main bottom 14 have a skeleton (or solid structure) for which the fill ratio is of order 100%.

The main bottom 14 comprises recesses 21 that are open towards the treatment chamber 5, and configured to receive, by socketing, portions of the lower edge 13, in particular the lower annular edge 13, of the main shell 12. In particular, the main bottom 14 comprises an outer rim 15, discontinuous in this specific example, projecting from the inner face 14b of the main bottom 14 into the treatment chamber 5 along the peripheral edge 17 of the main bottom 14. The main bottom 14 likewise comprises the portions of vertical wall 18 projecting from the inner face 14b of the main bottom 14 into the treatment chamber 5, and at least partially delimiting, with said outer rim 15, said recesses 21.

Preferably, the main shell 12 comprises an outer lower edge 13a and an inner lower edge 13b set back from said outer lower edge 13a, in particular at a distance dr, for example of order 1 mm or 2 mm. The inner lower edge 13b is configured to cooperate, by interlocking, with the receiving recesses 21. The outer lower edge 13a abuts on the outer rim 15 of the main bottom 14. The lower edge 13 of the main shell 12 thus cooperates with the main bottom 14 for their connection. The outer lower edge 13a of the main shell 12 abuts on the upper face of the outer rim 15, and preferably has a width dr of the same order as that of the outer rim 15 so that the outer surface 12a of the main shell 12 is prolonged by the outer face of the outer rim 15 without creating a protrusion at their junction. This arrangement makes it possible to perfectly position the main shell 12 relative to the main bottom 14.

Finally, the main shell 12 comprises securing through-openings 8, in this example six in number. These securing openings 8 are adjacent to the lower edge 13, in particular to the outer lower edge 13a. The main bottom 14 likewise comprises securing openings 19, in this example six in number, which are adjacent to the peripheral edge 17. Said securing openings 8 and 19, which are through-openings, are configured so as to enable the passage of at least one securing member (not shown), such as a suture yarn, through a securing opening 8 of the main shell 12 and a securing opening 19 of the main bottom 14. In this example, six securing members, for example staples or suture yarns, are disposed per pair of openings, comprising a securing opening 8 and a securing opening 19. The main shell 12 and the main bottom 14 are thus securely secured together once said at least one soft tissue and other elements have been placed in the treatment chamber 5. The first implantable device is optimised in terms of mass, degree of openings, and mechanical performance (impact resistance, fatigue resistant, etc.) which enables a reliable device, homogeneous growth of said at least one soft tissue, and controlled and improved patient comfort to be provided.

For example, for a volume of a treatment chamber 5 of 475 cm³, the mass of the main shell 12 is 38 g and the mass of the main bottom 14 is 9 g.

For example, for a volume of a treatment chamber 5 of 175 cm³, the mass of the main shell 12 is 19 g and the mass of the main bottom 14 is 6 g.

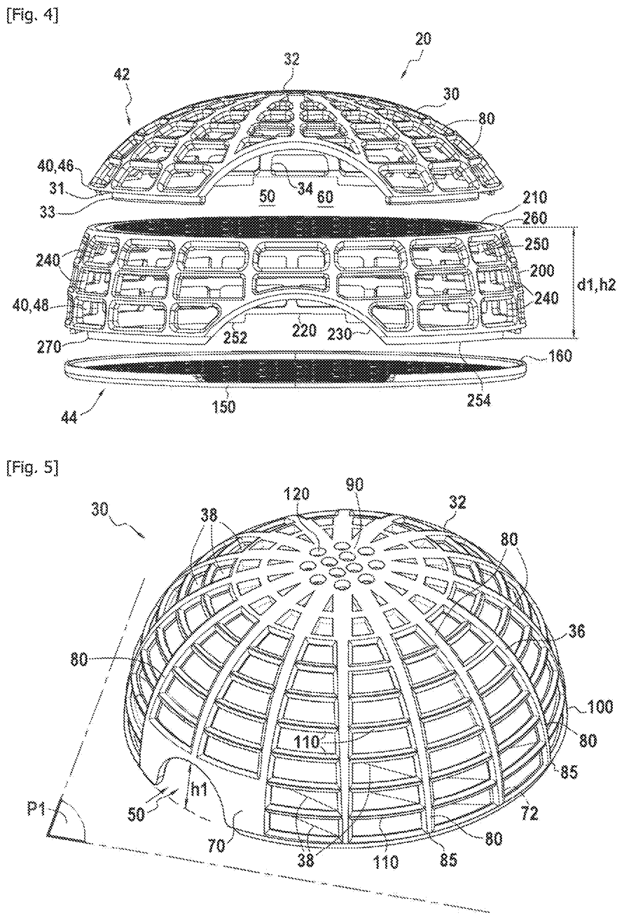

The second example of an implantable device 20, shown in FIG. 4, enables the formation of a treatment chamber having a volume greater than the volume of the treatment chamber of the first example device 10.

Advantageously, the device 10 comprises two lateral through-openings, one of which is visible in FIG. 1, in order to allow the placing of two vascular pedicles, one vascular pedicle being partially passed through one lateral through-opening.

This device 20 comprises a main shell 30 and a chamber 40 for treatment of a soft tissue. Said device 20 is further configured to receive at least one vascular pedicle using a first transverse opening 50 and a second transverse opening 60, each emerging in the treatment chamber 40 and on the outside of the main shell 30. The main shell 30 has an outer surface 32 and an inner surface 34 and comprises a set of through-openings 38 extending between said inner 34 and outer 32 surfaces and emerging thereon. Preferably, the ratio of the total surface area (mm²) of the through-openings 38, over the total surface area of the outer surface 32 of the main shell 30, is greater between 40% and 45%.

The structure of the main shell 30 has a fill ratio of order 100%.

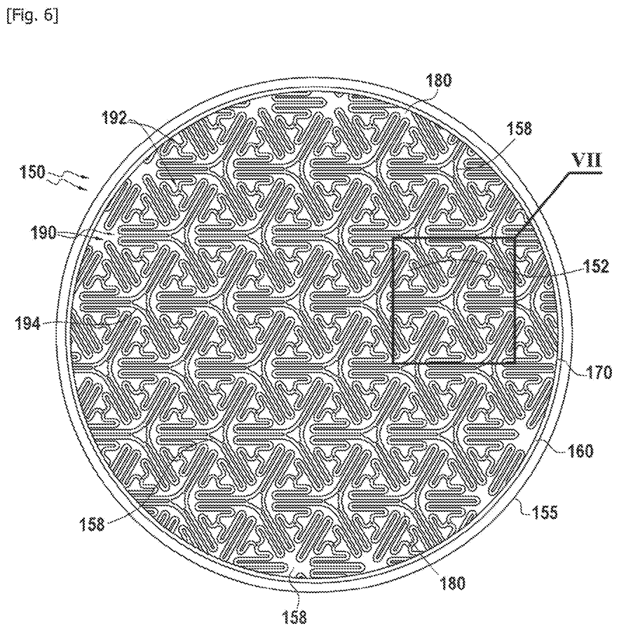

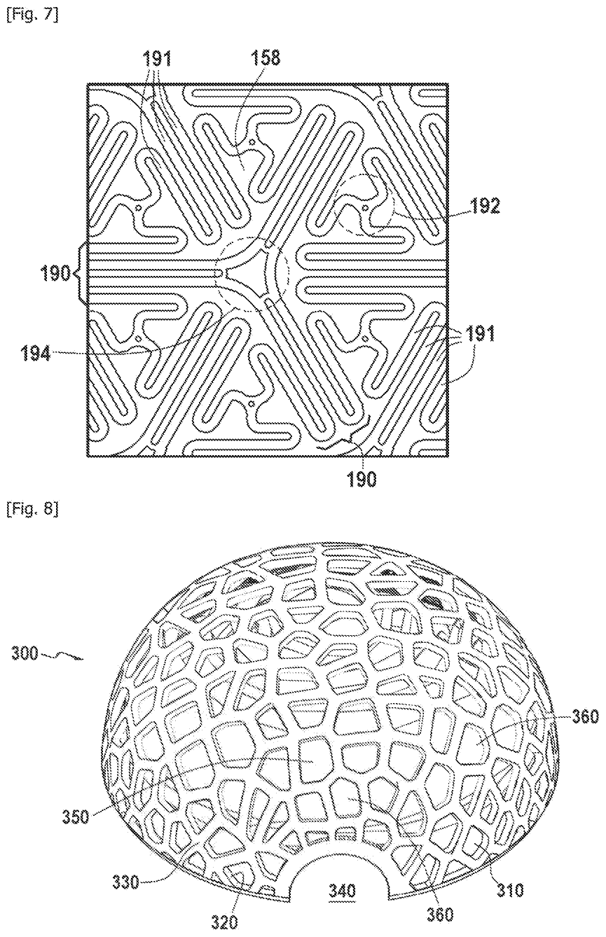

In the alternative shown in FIGS. 4 and 5, the main shell 30 comprises an assembly of arches 80, in this specific example 15 in number, extending between an apex of the main shell 30, and an annular transverse base 100 passing via some of the distal ends 85 of the arches 80. The annular base 100 is in a support plane P1. The distal ends of the arches 80 disposed above the first transverse opening 50 are not in the support plane P1, but spaced at a maximum height h1 from said plane P1, for the disposition of least one vascular pedicle.

The main shell 30 comprises transverse sections 110 that are, in particular, at least partially annular, extending between two adjacent arches 80. The main shell comprises a plurality of transverse sections 110 extending between neighbouring arches, said transverse sections 110 being spaced from one another. The main shell 30 may also comprise a set of through-openings 120 disposed at the apex 90.

The device 20 also comprises a main bottom 150 forming a rear portion 44 of the treatment chamber 40, the main shell 30 forming at least a front portion 42 of the treatment chamber 40.

The device 20 comprises an intermediate shell 200 having an upper receiving partition 210, subdividing the treatment chamber 40 into an upper treatment chamber 46 and a lower treatment chamber 48. Each of said upper and lower treatment chambers 46, 48 being configured to receive at least one vascular pedicle via first and second transverse through-openings 50, 60 for the upper treatment chamber 46 and via first and second transverses through-openings 220 and 230 for the lower treatment chamber 48.

The upper receiving partition 210 is configured so that when it is disposed in the treatment chamber 40, it is spaced apart from the main bottom 150 by a distance d1 greater than 0 mm, for example of order 2 mm to 8 mm, in order to at least partially delimit the lower treatment chamber 48. The intermediate shell 200 comprises through-openings 240 provided in its lateral partition 250 and emerging both on the inner 222 and outer 224 faces of said lateral partition 250. The lateral partition 250 projects from the lower face of the upper receiving partition 210 into the lower treatment chamber 48 and towards the main bottom 150.

The lateral partition 250 thus has a height h2 on the order of the distance d1.

In an embodiment, the ratio of the total surface area of the through-openings 240 emerging on the inner or outer face of the lateral partition 250, over the total surface area of the inner or outer face of the lateral partition 250, is of the same order as that developed above concerning the through-openings 38 in the main shell 30.

In an embodiment, the peripheral edge 31 of the main shell 30 comprises one or more projections (or lower annular edges) 33 configured to cooperate with a hollow receiving zone 260, such as a substantially annular recess, disposed on the perimeter of the upper receiving wall 210, for reversible securing between the main shell 30 and the intermediate shell 200.

In an embodiment, the lateral partition 250 of the intermediate shell 200 comprises one or more projections (or lower annular edges) 270 configured to cooperate with a receiving zone 160, such as a substantially annular recess, shown in FIG. 6 and disposed on the peripheral perimeter of the main bottom 150, for reversible securing between the intermediate shell 200 and the main bottom 150.

This receiving zone 160 is partially delimited, on the one hand, between the peripheral edge 170 projecting from an upper face 180 of the main bottom 150 towards the treatment chamber 40, in particular towards the lower treatment chamber 48, in particular set back from the outer edge 155 of the main bottom 150, and on the other hand the outer edge 155.

In an embodiment, the main bottom 150 shown in FIG. 6 is substantially planar, and comprises, on its support face 152, through-openings 158 defining an assembly of first patterns 190, repeated over the support surface 152, connected to one another via first connection structures 192 and second connection structures 194, likewise repeated on the support surface 152. The first pattern 190 comprises four segments 191 that are parallel with one another and spaced apart from one another by through-openings 158. In this specific example, for a same first pattern 190, two central segments 191 are secured to a second connection structure 194 while a first outer segment 191 is secured to a first connection structure 192, different from another first connection structure 192 to which a second outer segment 191 is secured. In this specific example, the first connection structures 192, and the second connection structures 194, are each secured to three first patterns 190. The first connection structures 192 have different shapes from the second connection structures 194.

The main bottom 150 is thus very flexible along its support surface 152 while being porous. In this specific example, the support surface of the upper receiving wall 210 is similar to the support surface 152.

The arrangement of the main bottom 150 can likewise be applied to the upper receiving wall 260.

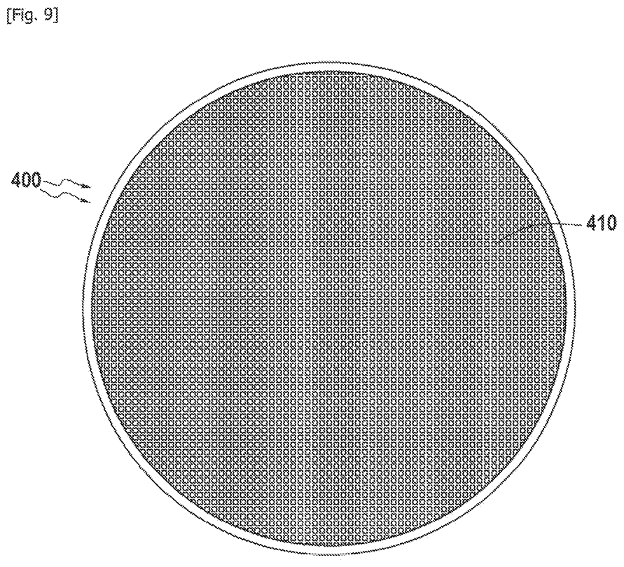

The main shell 12 or 30 can likewise be replaced by the main shell 300 shown in FIG. 8. The main shell 300 comprises through-openings 310 which are Voronoi polyhedra. The main shell 300 comprises two lateral through-openings 340 emerging in the treatment chamber 360, for the placing of at least one vascular pedicle.

The main bottom 150 or 14 can likewise be replaced by the main bottom 400 shown in FIG. 9, different from the main bottom 150 or 14, due to its support face 410 not comprising the first patterns and first and second connection structures, but multiple through-openings, in particular substantially parallelepiped through-openings. This support surface 410 is likewise very flexible and can deform in particular in its central region but not as much as the support face 152.

In operation, the implantable device is selected according to the desired volume of the treatment chamber. If the volume is large, for example greater than 400-500 cm3, a main shell, for example one of the shells 10, 30 or 300, is combined with an intermediate shell, for example the intermediate shell 200, and a main bottom, for example the main bottom 14, 150 or 400. If the desired volume of the treatment chamber is less than 400-500 cm3, a main shell, for example one of the shells 10, 30 or 300, is combined directly with a main bottom, for example the main bottom 14, 150 or 400. Then, an assembly comprising at least one porous layer support combined with at least one layer of cells chosen from adipocytes is disposed either in each of the upper and lower treatment chambers, or in the single treatment chamber. At least one vascular pedicle is combined with an assembly for supplying and growth of the soft tissue.

The porosity of each of the implantable devices according to the invention, connected to the through-openings provided in one or more walls of the one or more main and/or intermediate shells and/or the main bottom, promotes the growth of the soft tissue by homogeneous circulation of serous fluid. Moreover, the support surfaces are flexible and porous due to the through-openings that they comprise, thus promoting the circulation of serous fluid but also the placing of the assemblies disposed in the upper and lower treatment chambers in contact with one another, and/or with the implantation zone in the case of the main bottom. Finally, the mass of the devices is optimised while providing satisfactory mechanical performance through the use of a highly flexible (co)polymer such as defined in the present document.

1. An implantable device comprising a main shell and a chamber for treating at least one soft tissue, said device is further configured to receive at least one vascular pedicle, the main shell comprising a set of through-openings and being bioresorbable, wherein said main shell comprises at least one bioresorbable (co)polymer having an elongation at break greater than or equal to 200%.

2. The implantable device according to claim 1, wherein said bioresorbable copolymer is a copolymer of ε-caprolactone and L-, D- or DL-lactide.

3. The implantable device according to claim 1, wherein the main shell has an outer surface, and wherein a ratio of a total surface area (mm²) of the through-openings, over a total surface area of the outer surface of the main shell, is greater than or equal to 35%.

4. The implantable device according to claim 1, wherein the main shell has an outer surface, and wherein a ratio of a surface area of a solid outer surface of the main shell, over a total surface area of outer surface of the main shell, is greater than or equal to 40%.

5. The implantable device according to claim 1, wherein the main shell has a skeleton having a fill ratio greater than or equal to 70%.

6. The implantable device according to claim 1, wherein the implantable device comprises a main bottom having through-openings and forming a rear portion of a treatment chamber.

7. The implantable device according to claim 1, wherein said implantable device it comprises an intermediate shell comprising through-openings and an upper receiving partition subdividing a treatment chamber into an upper treatment chamber and a lower treatment chamber, said implantable device being configured so that each of said upper and lower treatment chambers is able to receive at least one vascular pedicle.

8. The implantable device according to claims 6 and 7, wherein said upper receiving partition is spaced apart from the main bottom by a distance d greater than 0 mm in order to at least partially delimit the lower treatment chamber.

9. The device according to claim 7, wherein the intermediate shell comprises a lateral partition projecting from a lower face of the upper receiving partition.

10. The device according to claim 6, wherein the main bottom comprises one or more recesses, open towards the treatment chamber, and configured to receive, in particular by interlocking, a portion or portions of a lower edge of the main shell or of an intermediate shell.

11. The device according to claims 6 and 10, wherein the main bottom comprises:
a continuous or discontinuous outer rim, projecting from an inner face of the main bottom into the treatment chamber, and
one or more portions of vertical wall projecting from the inner face of the main bottom into the treatment chamber, and at least partially limiting, with said outer rim, said recess or recesses.

12. The device according to claim 10, wherein the main shell or the intermediate shell comprises an outer lower edge, and an inner lower edge set back from said outer lower edge.

13. The device according to claim 12, wherein the outer lower edge abuts on an outer rim of the main bottom.

14. The device according to claim 6, wherein the main shell comprises one or more securing openings, and the main bottom comprises one or more securing openings, said securing openings being configured so as to allow the passage at least one securing member through a securing opening of the main shell and a securing opening of the main bottom.

15. The implantable device according to claim 1, wherein at least one portion of the through-openings of the main shell are Voronoi polyhedra.

16. The implantable device according to claim 1, wherein the main shell comprises at least two vertical arches-Egg and transverse sections, in particular at least partially annular, extending between said vertical arches and connected thereto.

17. The implantable device according to claim 6, wherein the main bottom comprises through-openings arranged to define a plurality of first patterns connected to one another by first connection structures.

18. The device according to claim 17, wherein the first patterns comprise one or more segments at least one or more segments of each first pattern being secured to a first connection structure.

19. The device according to claim 17, wherein the first connection structures are connected to at least three distinct first patterns.

20. The device according to claim 18, wherein the main bottom comprises second connection structures, different from the first connection structures, each of the second connection structures being connected to at least two segments of a same first pattern.

21. The device according to claim 20, wherein at least one of the second connection structures is connected to at least three distinct first patterns.

22. A method for manufacturing an implantable device according to claim 1, wherein it comprises a step of additive or subtractive manufacturing, of said main shell, with at least one yarn comprising said (co)polymer having an elongation at break greater than or equal to 200%.
